(19) United States
(12) Patent Application Publication
(10) Pub. No.: US 2010/0284927 A1
(43) Pub. Date: Nov. 11, 2010

(54) USE OF MRI CONTRAST AGENTS FOR EVALUATING THE TREATMENT OF TUMORS

(75) Inventors: Zheng-Rong Lu, Salt Lake City, UT (US); Yi Feng, Las Vegas, NV (US); Xueming Wu, Salt Lake City, UT (US)

Correspondence Address: GARDNER GROFF GREENWALD & VILLANUEVA. PC, 2018 POWERS FERRY ROAD, SUITE 800, ATLANTA, GA, 30339, US

(73) Assignee: University of Utah Research Foundation, Salt Lake City, UT (US)

(21) Appl. No.: 12/738,856

(22) PCT Filed: Oct. 23, 2008
(86) PCT No.: PCT/US08/80908

§ 371 Date: Jul. 19, 2010

Related U.S. Application Data
(60) Provisional application No. 60/982,878, filed on Oct. 26, 2007.

(51) Int. Cl. A61K 49/10

(52) U.S. Cl. 424/9.2

(57) ABSTRACT

Described herein are methods for using macromolecular MRI contrast agents to evaluate the effectiveness of anti-cancer treatments. The methods take advantage of MRI for evaluating more specifically and accurately one or more tumor properties of the tumor in response to a particular treatment. Ultimately, the 5 methods described herein help evaluate the effectiveness of the anti-cancer treatment over time.

### CROSS REFERENCE TO RELATED APPLICATIONS

This application claims priority upon U.S. provisional application Ser. No. 60/982,878, filed Oct. 26, 2007. This application is hereby incorporated by reference in its entirety for all of its teachings.

### ACKNOWLEDGEMENT

This invention was made with government support under grant nos. R33 CA095873 and R01 EB00489 awarded by the National Institutes of Health. The Government has certain rights to this invention.

### BACKGROUND

Cancer is the second leading cause of death in the United States. With 218,890 estimated new cases and 27,050 estimated deaths, breast cancer is the leading cause of morbidity and mortality in women. Lumpectomy (surgical excision), which is the primary treatment for breast cancer, has a drawback of disfigurement that remains as a significant psychological barrier.

MRI is an attractive non-invasive imaging modality for diagnosis and treatment of tumors due to its versatility (e.g., target localization, treatment planning, instrument visualization, online temperature monitoring, assessment of efficacy, and follow up). The use of MR guidance increases safety and efficacy of anti-cancer treatments. MRI-guided interstitial laser ablation has been shown to be an effective way of ablating breast carcinoma with a size of less than 1 cm (Korourian S, Klimberg S, Henry-Tillman R, et al. “Assessment of proliferating cell nuclear antigen activity using digital image analysis in breast carcinoma following magnetic resonance-guided interstitial laser photocoagulation” Breast J 2003; 9:409-413). Contrast enhanced MRI is superior to diffusion weighted and T2 weighted images in assessing the boundaries of tissue necrosis after anti-cancer treatment (Huang Z, Haider M A, Kraft S, et al. “Magnetic resonance imaging correlated with the histopathological effect of Pd-bacteriopheophorbide (Tookad) photodynamic therapy on the normal canine prostate gland” Lasers Surg Med 2006; 38:672-681: van Furth W R, Laughlin S, Taylor M D, et al. “Imaging of murine brain tumors using a 1.5 Tesla clinical MRI system” Can J Neurol Sci 2003; 30:326-332).

Angiogenesis, the process of new vessels growth, is critical for a tumor to grow beyond a few millimeters. Anti-angiogenic agents have been developed and tested in clinical trials on cancer patients. To test the tumor response to anti-cancer treatment, the traditional way to evaluate the response of the tumor to the drug is to measure the tumor size. However, this approach is not ideal for detecting vascular changes after treatment with anti-angiogenic agents because (1) the tumor regression does not necessarily correlate to the corresponding vascular effect, and (2) the change in tumor size might not happen immediately after changes in the vasculature. Therefore, it would be desirable to have techniques for evaluating the effectiveness of an anti-cancer treatment using non-invasive techniques. MRI could evaluate the presence and size of the residual tumor more sensitively, specifically, and accurately.

### SUMMARY

Described herein are methods for using macromolecular MRI contrast agents to evaluate the effectiveness of anti-cancer treatments. The methods take advantage of MRI for evaluating more specifically and accurately one or more tumor properties of the tumor in response to a particular treatment. Ultimately, the methods described herein help evaluate the effectiveness of the anti-cancer treatment over time. The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows 2D SE images of tumors enhanced by Gd(DTPA-BMA) (8 min post contrast agent injection) and GDCC-40 (16 min post contrast agent injection). Arrows point to the control tumor and dotted arrows point to the LA treated tumor.

FIG. 2 shows sample time curves of tumor rim and muscle signal intensity (SI), where the ratios were enhanced by GDCC-40 and Gd(DTPA-BMA).

FIG. 3 shows 2D spin echo MR images using various doses of GDCC-40. White arrows point to the untreated tumor in each image.

FIG. 4 shows the enhancement of untreated tumor by 0.01, 0.025, and 0.05 mmol-Gd/kg GDCC-40, respectively. The asterisk indicates statistically different from other doses.

FIG. 5 shows the SI time curves for blood and untreated tumor enhanced by GDCC-40 at 0.01, 0.025, and 0.05 mmol-Gd/kg, respectively.

FIG. 6 shows PV and PS mapping for MDA-MB-231 tumor obtained by DCE-MRI using GDCC-40.

FIG. 7 shows 2D SE MR images of tumor bearing mice 4 hr after treatment with DyeLA (A) and LA (B), before and 15 min after GDCC-40 injection. Solid arrows point to the bright area of treated tumors, indicating the inflammation caused by thermal damage. Dotted arrows point to the untreated tumor.

FIG. 8 shows 2D SE images pre- and 15 min post-contrast agent injection 4 hr (A) and 7 days (B) with parameter mapping of PV, FLR and PS after treatment.

FIG. 9 shows the SI of blood and untreated tumor enhanced by GDCC-40 at 0.0025 mmol/kg dose.

FIG. 10 shows the effect of Avastin treatment on tumor growth with the use of the HT-29 colon cancer cell line. The growth speed after treatment became much slower than that before treatment.

FIG. 11 shows representative contrast enhancement-time curves for GDCC40K (A) and Omniscan (B) before, 36 h and 7 days after administration of Avastin (obtained from the same tumor reported in FIG. 12).

FIG. 12 is a graph showing changes in K^{trans} (A) and f_{PV} (B) for GDCC and Omniscan before and after Avastin administration.

FIG. 13 shows K^{trans} and f_{PV} color maps obtained from the same tumor slices reported in FIG. 2, where (A) is from the tumor of GDCC40K injection group and (B) is from the tumor of Omniscan injection group.

### DETAILED DESCRIPTION

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a contrast agent” includes mixtures of two or more such agents, and the like.

“Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or can not be substituted and that the description includes both unsubstituted lower alkyl and lower alkyl where there is substitution.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

The methods described herein generally involve the use of MRI contrast agents in combination with MRI for evaluating the effectiveness of a cancer therapy or treatment. The methods permit the noninvasive monitoring of a tumor during the treatment of the tumor before, during, and after treatment of the tumor. In particular, the methods can monitor quantitatively one or more properties of the tumor over time. The nature of the property to be evaluated will depend upon the particular therapy or treatment selected. Details regarding the tumor properties that can be evaluated herein are described below.

In one aspect, the method involves:
a. administering to the subject a contrast agent, wherein the contrast agent comprises a biodegradable macromolecular Gd(III) complex;
b. evaluating a tumor property by magnetic resonance imaging to establish a baseline of the tumor property;
c. treating the tumor in the subject;
d. evaluating the tumor property after step (c) by magnetic resonance imaging; and
e. comparing the results of step (d) with the baseline in step (b) to determine the effectiveness of the tumor treatment.
   In this aspect, the first two steps involve administering a contrast agent to the subject prior to the treatment of the tumor, and obtaining a baseline value of one or more tumor properties. For example, the signal intensity of the contrast agent at a region of interest in the tumor is measured prior to treatment using magnetic resonance imaging. The term ‘region of interest” is defined herein as a specific region located at the tumor where baseline and subsequent signal intensities are detected and quantified. In one aspect, the region of interest is the periphery of the tumor. In another aspect, the region of interest is the whole tumor. The region of interest can vary depending upon the treatment selected.

Contrast agents useful herein are disclosed in U.S. Pat. No. 6,982,324, which are incorporated by reference in their entirety. The contrast agents are generally biodegradable so that over time they degrade into smaller molecules or fragments that can be readily removed from subject via the circulatory system. For these reasons, the macromolecular Gd(III) complex useful herein is a large molecule so that once incorporated into the tumor cells it is not readily removed from the cells via the circulatory system. The contrast agents also exhibit little to toxicity to healthy cells.

In one aspect, contrast agents useful herein are represented by the following generic formulae:

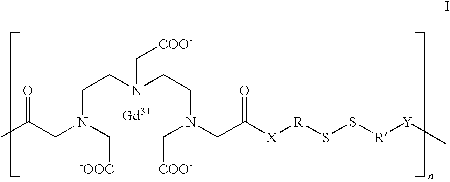

wherein R and R′ comprises, independently, a C₁ to C₁₈ alkyl group, a substituted alkyl group, an unsubstituted aryl group or an aryl group substituted with one or more functional groups comprising an alkyl group, an aryl group, a polyethylene glycol, a saccharide, an amino acid, a peptide, a protein, a peptide conjugate, or a protein conjugate;

X and Y are, independently, O and NH; and

n is an integer between 2 and 10,000;

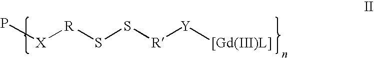

wherein R and R′ comprises, independently, a C₁ to C₁₈ alkyl group, a substituted alkyl group, an unsubstituted aryl group or an aryl group substituted with one or more functional groups comprising an alkyl group, an aryl group, a polyethylene glycol, a saccharide, an amino acid, a peptide, a protein, a peptide conjugate, or a protein conjugate;

X and Y comprises, independently, an amide group, an ester group, a urea group, a thiourea group, a carbonate group, a carbamate group, an ether bond, or a thioether bond;

P comprises a water soluble polymer chain, a dendrimer, a polysaccharide, a peptide, a protein, a polymer-peptide conjugate, or a polymer-protein conjugate; n is an integer between 2 and 10,000; and

L comprises diethylenetriaminepentaacetate (DTPA) or its derivatives; 1,4,7,10-tetraazadodecanetetra-acetate (DOTA) or its derivatives; 1,4,7,10-tetraazadodecane-1,4,7-triacetate (DO3A) or its derivatives, or a chelating ligand;

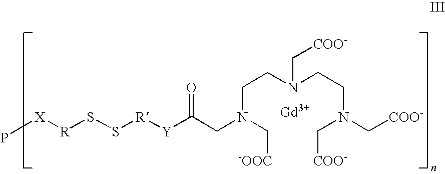

wherein R and R′ comprises, independently, a C₁ to C₁₈ alkyl group, a substituted alkyl group, an unsubstituted aryl group or an aryl group substituted with one or more functional groups comprising an alkyl group, an aryl group, a polyethylene glycol, a saccharide, an amino acid, a peptide, a protein, a peptide conjugate, or a protein conjugate;

X comprises an amide group, an ester group, a urea group, a thiourea group, a carbonate group, a carbamate group, an ether bond, or a thioether bond;

Y comprises O or NH;

n is an integer between 2 and 10,000; and

P comprises a water soluble polymer chain, a dendrimer, a polysaccharide, a peptide, a protein, a polymer-peptide conjugate, or a polymer-protein conjugate;

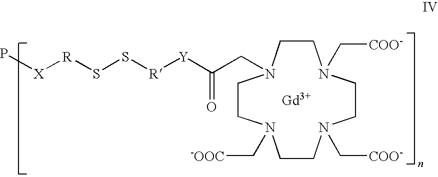

wherein R and R′ comprises, independently, a C₁ to C₁₈ alkyl group, a substituted alkyl group, an unsubstituted aryl group or an aryl group substituted with one or more functional groups comprising an alkyl group, an aryl group, a polyethylene glycol, a saccharide, an amino acid, a peptide, a protein, a peptide conjugate, or a protein conjugate;

X comprises an amide group, an ester group, a urea group, a thiourea group, a carbonate group, a carbamate group, an ether bond, or a thioether bond;

Y comprises O or NH;

n is an integer between 2 and 10,000; and

P comprises a water soluble polymer chain, a dendrimer, a polysaccharide, a peptide, a protein, a polymer-peptide conjugate, or a polymer-protein conjugate;

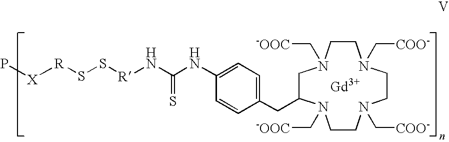

wherein R and R′ comprises, independently, a C₁ to C₁₈ alkyl group, a substituted alkyl group, an unsubstituted aryl group or an aryl group substituted with one or more functional groups comprising an alkyl group, an aryl group, a polyethylene glycol, a saccharide, an amino acid, a peptide, a protein, a peptide conjugate, or a protein conjugate;

X comprises an amide group, an ester group, a urea group, a thiourea group, a carbonate group, a carbamate group, an ether bond, or a thioether bond;

n is an integer between 2 and 10,000; and

P comprises a water soluble polymer chain, a dendrimer, a polysaccharide, a peptide, a protein, a polymer-peptide conjugate, or a polymer-protein conjugate;

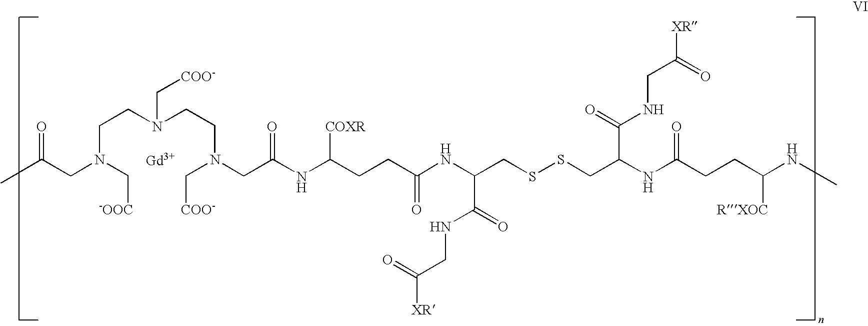

wherein R, R′, R″ and R′″ comprise, independently, a C₁ to C₁₈ alkyl group, a substituted alkyl group, an unsubstituted aryl group or an aryl group substituted with one or more functional groups comprising an alkyl group, an aryl group, a polyethylene glycol, a saccharide, an amino acid, a peptide, a protein, a peptide conjugate, or a protein conjugate;

X comprises O or NH; and

n is an integer between 2 and 10,000.

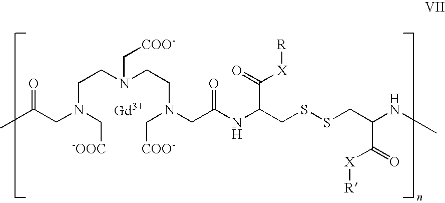

wherein R and R′ comprises, independently, hydrogen, a C₁ to C₁₈ alkyl group, a substituted alkyl group, an unsubstituted aryl group or an aryl group substituted with one or more functional groups comprising an alkyl group, an aryl group, a polyethylene glycol, a saccharide, an amino acid, a peptide, a protein, a peptide conjugate, or a protein conjugate;

X comprises O or NH; and

n is an integer between 2 and 10,000.

In other aspects, the contrast agents useful herein are represented by the following formulae:

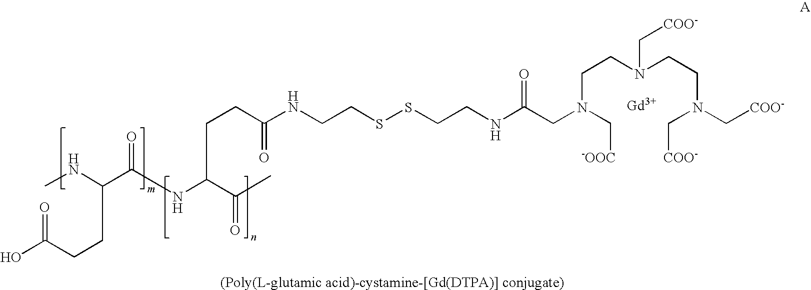

wherein n is an integer between 2 and 10,000; and

wherein m is an integer between 0 and 10,000;

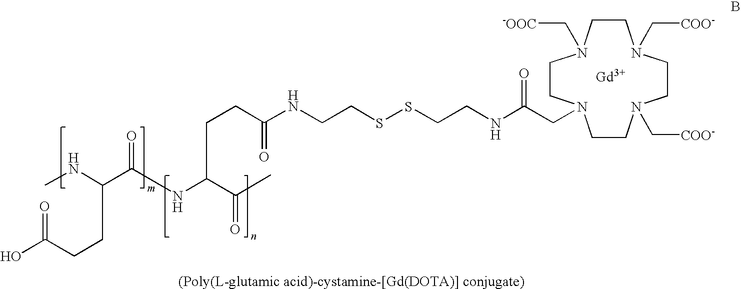

wherein n is an integer between 2 and 10,000; and

wherein m is an integer between 0 and 10,000;

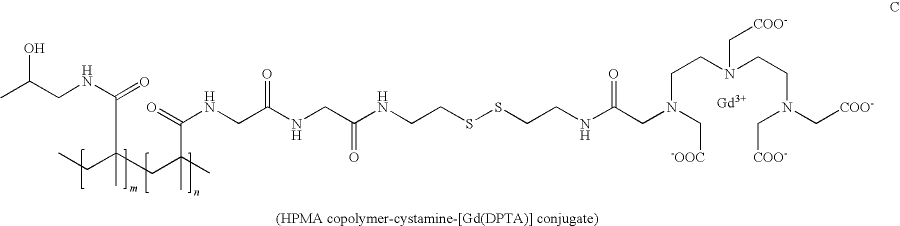

wherein n is an integer between 2 and 10,000; and

wherein m is an integer between 0 and 10,000;

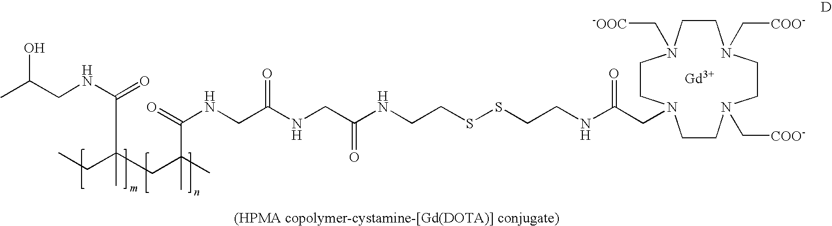

wherein n is an integer between 2 and 10,000; and

wherein m is an integer between 0 and 10,000;

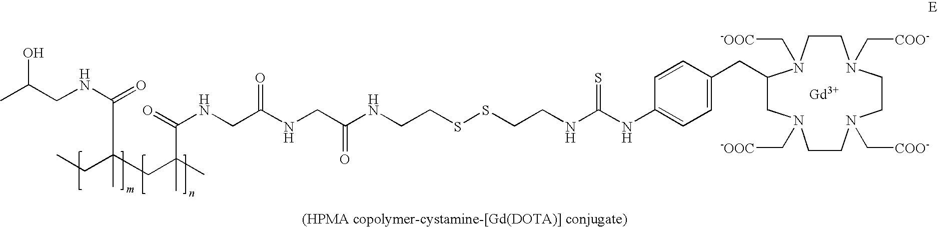

wherein n is an integer between 2 and 10,000; and

wherein m is an integer between 0 and 10,000;

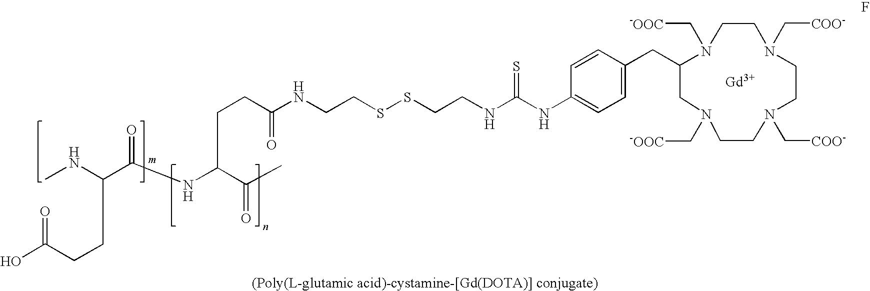

wherein n is an integer between 2 and 10,000; and

wherein m is an integer between 0 and 10,000;

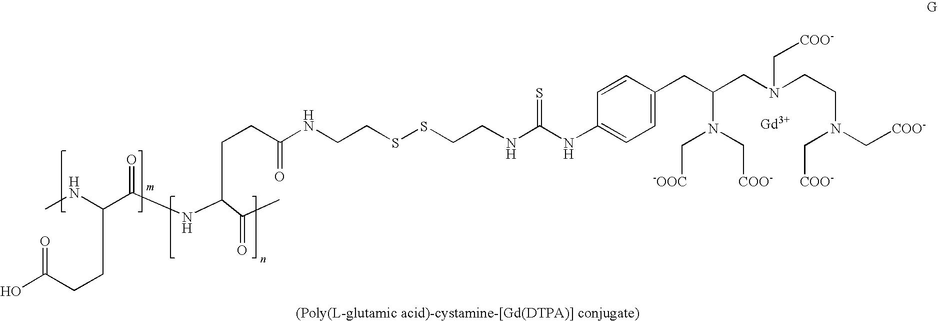

wherein n is an integer between 2 and 10,000; and

wherein m is an integer between 0 and 10,000;

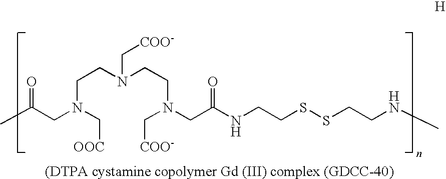

wherein n is an integer between 2 and 10,000;

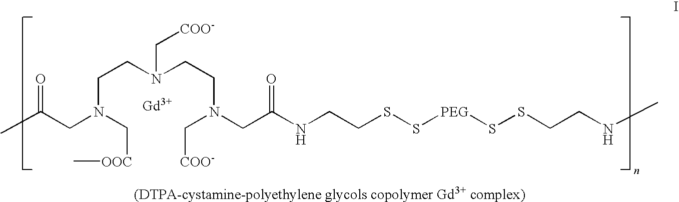

wherein n is an integer between 2 and 10,000;

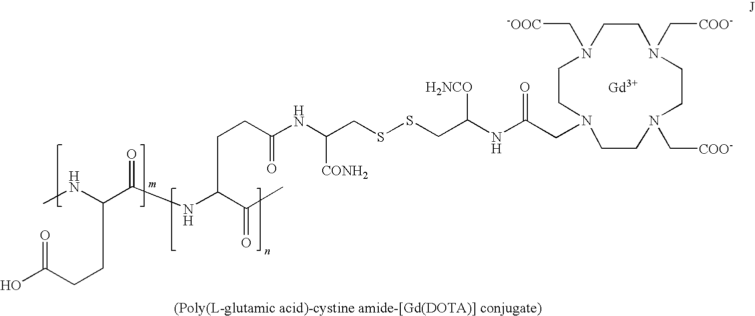

wherein n is an integer between 2 and 10,000; and

wherein m is an integer between 0 and 10,000; and

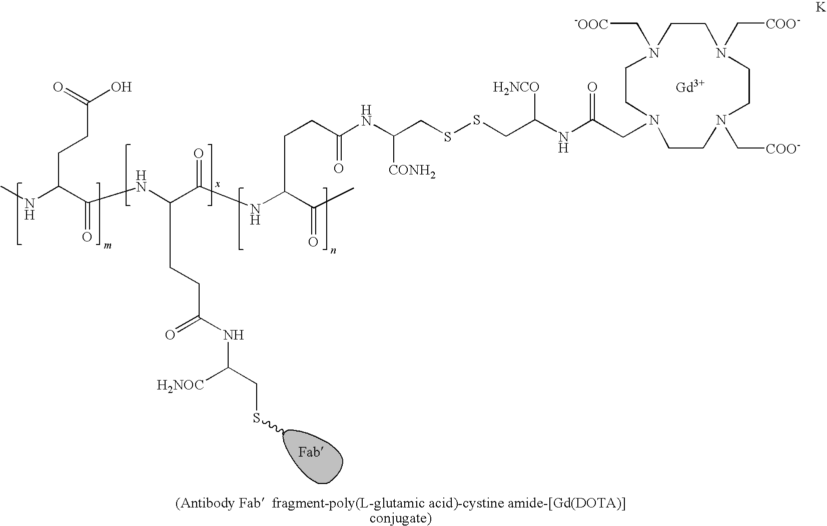

wherein n is an integer between 2 and 10,000; and

wherein x is an integer between 1 and 20; and

wherein m is an integer between 0 and 10,000.

In certain aspects, macromolecular Gd(III) complex can include one or more polyethylene glycol (PEG) groups. For example, one or more PEG groups can be incorporated into the P, R, and/or R′ groups in formulae I-VII. In one aspect, the PEG groups independently have molecular weights of at least about 50 Daltons, at least about 500 Daltons, at least about 1000 Daltons, or at least about 2000 Daltons. In other aspects, the molecular weights of PEG groups are independently from about 50 Daltons to about 50,000 Daltons.

The treatment of the tumor involves the administration of one or more anti-cancer agents to the subject. The term “anti-cancer agent” as defined herein is any compound or drug that inhibits the growth of a tumor or reduces the growth rate of the tumor. For example, photosensitizers can be administered so that upon exposure to energy, the photosensitizer accumulated in the tumor is activated and kills the cancer cells. In one aspect, thermal ablation, or radiofrequency ablation, cryoablation, high intensity focused ultrasound ablation (HIFU), or laser ablation can be used to activate the photosensitizer. The techniques disclosed in U.S. Pat. Nos. 5,829,448; 5,736,563, and 5,630,996 regarding photodynamic therapy can be used herein.

In other aspects, the anti-cancer agent includes one or more anti-angiogenic agents. It has been established that angiogenesis (i.e., the development of neovasculature from endothelial cells), is crucial for tumor development, growth and metastasis. Several growth factors have been identified as possible regulators of angiogenesis. One of the most important positive regulators of angiogenesis is vascular endothelial growth factor (VEGF). Accordingly, various inhibitors of VEGF have been developed to block tumor angiogenesis including Avastin (Bevacizumab, Genentech, South San Francisco, Calif.), a humanized anti-VEGF monocolonal antibody.

As described above, a baseline value of one or more tumor properties is established at a particular region of interest at the tumor. After administration of the contrast agent and the anti-cancer agent, the one or more tumor properties are evaluated by MRI to determine the effectiveness of the treatment. For example, if MRI indicates that the size of the tumor has reduced after administration of the anti-cancer agent, then it can be established that the selected treatment is effective in treating the tumor.

In one aspect, the methods are useful in evaluating the effectiveness of anti-angiogenic agents. The ability to evaluate the efficacy of anti-angiogenic agents is very challenging. Current methods cannot assess early microvasculature changes within the tumor. The methods described herein permit the quantification of a number of tumor microvascular characteristics such as, for example, fractional tumor blood volume (i.e., the density of vasculature in the tumor tissue), and the ability of the contrast agent to cross the tumor vasculature (e.g., flow leakage rate, permeability surface area, vascular permeability, or transfer constant). In certain aspects, when the effectiveness of an anti-angiogenic agent is to be evaluated, the region of interest at the tumor is the periphery of the tumor. Not wishing to be bound by theory, higher amounts of blood vessels are present toward the rim of the tumor when compared to the center of tumor. Thus, if the signal intensity of the periphery of the tumor is lower after the administration of the anti-angiogenic agent when compared to the baseline value prior to the administration of the anti-angiogenic agent, this is an indication that the anti-angiogenic agent is effective in reducing one or more neovascular properties within the tumor. In other words, a decrease in signal intensity after administration of the anti-angiogenic agent indicates that less contrast agent is present in the tumor, which is due to a change in one or more neovascular properties within the tumor.

The methods described herein can qualitatively evaluate the effectiveness of the treatment (e.g., visual decrease in intensity of the contrast agent present in the tumor). In other aspects, changes in one or more tumor properties can be quantified to further evaluate the effectiveness of the treatment. Methods for quantitatively evaluating neovasculature properties of a tumor are provided in the Examples. The tumor property can be evaluated as many times as needed over time in order to evaluate the effectiveness of the treatment. For example, after administration of the anti-cancer agent, one or more tumor properties can be evaluated by MRI. Subsequent analysis of the tumor property by MRI can be performed in order to evaluate the effectiveness of the treatment over time. This is particularly useful in the treatment of the cancer and identifying the best treatment options for the subject.

The methods described herein use contrast agents that can be readily detected and quantified by MRI. In one aspect, dynamic contrast-enhanced MRI (DCE-MRI) is used herein. DCE-MRI is functionally more useful than traditional snapshots of contrast enhanced MR. In addition to taking snap shots of MR images at specified time points before and after the injection of the contrast agent, dynamic contrast enhanced (DCE) MRI can also provide a time course of the signal enhancement (i.e., contrast agent uptake by the tissue), which can provide qualitative and quantitative information about one or more vascular properties of the tumor.

The amount of contrast agent administered to the subject can vary depending upon the selection of the tumor property that is to be evaluated, the MRI technique, and the type of treatment. In one aspect, the contrast agent is administered to the subject in an amount of 0.001 to 0.5 mmol-Gd/kg. In other aspects, the amount of contrast agent is from 0.001 to 0.5 mmol-Gd/kg, 0.001 to 0.4 mmol-Gd/kg, 0.001 to 0.3 mmol-Gd/kg, 0.001 to 0.2 mmol-Gd/kg, 0.001 to 0.1 mmol-Gd/kg, 0.01 to 0.1 mmol-Gd/kg, 0.025 to 0.075 mmol-Gd/kg, or about 0.05 mmol-Gd/kg. The contrast agent and the anti-cancer agent can be administered by any method and/or applicator known in the art. In one aspect, contrast agent and the anti-cancer agent can be administered by injection, such as by a syringe, needleless injector, and/or the like. In other aspects, contrast agent and the anti-cancer agent can be administered orally.

The contrast agent and anti-cancer drug can be administered independently or collectively as a pharmaceutical composition. Pharmaceutical compositions described herein can be formulated in any excipient the biological system or entity can tolerate. Examples of such excipients include, but are not limited to, water, saline, Ringer's solution, dextrose solution, Hank's solution, and other aqueous physiologically balanced salt solutions. Nonaqueous vehicles, such as fixed oils, vegetable oils such as olive oil and sesame oil, triglycerides, propylene glycol, polyethylene glycol, and injectable organic esters such as ethyl oleate can also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosol, cresols, formalin and benzyl alcohol.

Pharmaceutical carriers are known to those skilled in the art. These most typically would be standard carriers for administration to humans, including solutions such as sterile water, saline, and buffered solutions at physiological pH. Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally). In the case of contacting cells with the dendrimers described herein, it is possible to contact the cells in vivo or ex vivo.

Preparations for administration include sterile aqueous or non-aqueous solutions, suspensions, and emulsions. Examples of non-aqueous carriers include water, alcoholic/aqueous solutions, emulsions or suspensions, including saline and buffered media. Parenteral vehicles, if needed for collateral use of the disclosed compositions and methods, include sodium chloride solution, Ringer's dextrose, dextrose and sodium chloride, lactated Ringer's, or fixed oils. Intravenous vehicles, if needed for collateral use of the disclosed compositions and methods, include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like. It is understood that any given particular aspect of the disclosed compositions and methods can be easily compared to the specific examples and embodiments disclosed herein, including the non-polysaccharide based reagents discussed in the Examples. By performing such a comparison, the relative efficacy of each particular embodiment can be easily determined. Particularly preferred compositions and methods are disclosed in the Examples herein, and it is understood that these compositions and methods, while not necessarily limiting, can be performed with any of the compositions and methods disclosed herein.

### Examples

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### I. Validation of Laser Based Photothermal Therapies and BMCA Enhanced MRI to Assess the Efficacy

### Materials and Methods

Gd-(DTPA-BMA) (Omniscan, gadodiamide) was obtained from Nycomed Inc. (Princeton, N.J., USA). Ketamine and xylazine were obtained from Ben Venue Labs (Bedford, Ohio, USA) and Vedco Inc. (St. Joseph, Mo., USA), respectively. Fractions of (Gd-DTPA)-cystamine copolymers (GDCC-40) with number average molecular weight of 39 KDa and weight average molecular weight of 43 KDa were similarly prepared as previously described (Lu Z R, Parker D L, Goodrich K C, Wang X, Dalle J G, Buswell H R. “Extracellular biodegradable macromolecular gadolinium(III) complexes for MRI” Magn Reson Med. 2004; 51:27-34).

### Animal Tumor Model

Female athymic nude mice (5-6 weeks old, Frederick, M D, National Cancer Institute) were cared for under the guidelines of a protocol approved by the University of Utah Institutional Animal Care and Use Committee. The MDA-MB-231 human breast cancer cell line was purchased from the American Type Culture Collection (ATCC, Manassas, Va.) and cultured in Leibovitz's L-15 medium with 2 mM L-glutamine and 10% FBS at 37° C. in a humidified atmosphere of 5% CO₂. 5×10⁶ cells in a mixture of 50 μL medium and 50 μL Matrigel (Becton-Dickinson, Franklin Lakes, N.J.) were inoculated subcutaneously on both sides of the hips of each mouse. Each implantation generated a tumor. When the tumor size reached about 100 mm³ or 500 mm³, they were subjected to treatment. After the treatment the mice were kept in individual cage to avoid the chewing of the treated tumor by other mouse.

The size of the tumors were monitored regularly. Size=π/6×D1×D2×D3 (π=3.14, D1, D2, and D3 are the tumor length, width, and thickness measured by digital caliber, respectively). Each tumor size was normalized to the size at the treatment day (Day 0): ratio=Size_{Day 7 or day 12}/Size_{Day 0}.

### Characterization of Laser Energy Deposition

DenLaser 800 (Cao Group Inc., Salt Lake City, Utah) was used for both LA and DyeLA. This Diode laser operates at λ=810 nm with the aiming beam at λ=650 nm. To calculate the dose of laser light, the diameter of the laser light spot at λ=650 nm on a piece of paper was measured at a distance of 0.5, 1, 2, or 3 cm. To conceptually simulate the dye enhanced laser energy absorption, 1 ml of 0% or 0.4% Trypan Blue Stain (BioWhittaker, Walkersville, Md) was added into wells of 96-well plate. The laser fiber tip was maintained at 5 mm away from the bottom of one of the wells and laser energy at 1 or 5 W was applied. The temperature was measured using a thermometer after 5 min of laser irradiation.

### Photothermal Therapies

To modify the radial temperature distribution and shift maximum temperature into deeper tissue, cold water was dripped from the top of the tumor for cooling purposes for both LA and DyeLA. Two groups of tumor were used in this study: 1) big tumor (500 mm³), and 2) small tumor (100 mm³) The detailed parameters of treatment are listed in Table 1 (big tumor group) and Table 2 (small tumor group).

For DyeLA, 1.5% DI water solution of indocyanine green (ICG) (Sigma, St. Louis, Mo.) was injected intra-tumorally 4 hr before the treatment, The volume was approximately 100 uL for 500 mm³ and 30 uL for 100 mm³ tumors, respectively. The fiber tip was maintained at a 5 mm distance from the skin overlying the tumor.

For LA, the laser fiber was introduced using a 25 G needle. For group 2 and 3 in Table 1, laser ablations were applied twice: the first one along the longest axis of the tumor and then the second one perpendicular to the first one. For group 4 and 5, instead of one ablation each direction, two ablations were applied in parallel about 3 mm apart for each direction, giving a total of 4 ablations for each tumor. The application time of the laser was evenly distributed among all ablations.

TABLE 1
Parameters for treatment of big tumors (500 mm³).
Number of animals is 6 for each group.

| | | | Dye | | |
|---|---|---|---|---|---|
| Group | LA | DyeLA | Vol. | Cooling | Light dose |
| 1 | | | | | 0 |
| 2 | 1 W; 10 min | | | No | 600 J |
| 3 | 2 W; 10 min | | | No | 1200 J |
| 4 | 2 W; 5 min | | | No | 600 J |
| 5 | 2 W; 5 min | | | Yes | 600 J |
| 6 | | 2.5 W; 10 min | 100 uL | No | 4.6 KJ/cm² |
| 7 | | 5 W; 10 min | 100 uL | Yes | 9.2 KJ/cm² |

TABLE 2
Parameters for treatment of small tumors (100 mm³).

| | | Dye | | | |
|---|---|---|---|---|---|
| Group | Light dose | injected | Cooling | Light dose | No. of mice |
| Control | | | | 0 | 6 |
| LA | 2 W, 6 min | | Yes | 720 J | 6 |
| DyeLA | 5 W; 12 min | 30 uL | Yes | 11.1 KJ/cm | 10 |

### MRI Scan Procedure

The mice were anesthetized by the intraperitoneal administration of a mixture of ketamine (90 mg/kg) and xylazine (10 mg/kg). Tumor bearing mice were subjected to MR scan using a Siemens Trio 3T scanner 7 days after treatment using Gd-(DTPA-BMA) and 12 days after treatment using GDCC-40. Both contrast agents were injected intravenously via tail vein cannulization. The dose of Gd-(DTPA-BMA) was 0.1 mmol-Gd/kg and that of GDCC-40 was 0.05 mmol-Gd/kg.

2D spin echo (SE) images were acquired before contrast agent injection and at the end of DCE-MRI with the following parameters: TR/TE=400/10 ms, α=90, 0.4×0.4×2 mm, average=2, acquisition time=61 sec. 2D fast low angle shot pulse sequence (2D FLASH) was used for DCE-MRI with the following parameters: TR/TE=104/4.46 ms, α=30, 0.5×0.5×1.5 mm, average=1, acquisition time=11 sec, and a total of 10 slices per scan. The system body coil was used for RF excitation and a human wrist coil was used for RF reception. 2D FLASH images were repetitively acquired for 8 min for Gd-(DTPA-BMA) or 16 min for GDCC-40. Contrast agent was injected 45 sec after the 2D FLASH started in order to acquire 4 scans for the calculation of baseline signal intensity (SI). Shorter total scan time was used for Gd-(DTPA-BMA) because the kinetics of tumor enhancement by Gd-(DTPA-BMA) is faster than that by GDCC-40.

### Image Analysis

SI of the untreated whole tumor pre and post contrast enhancement in 2D SE images was obtained using Osirix (http://homepage.mac.com/rossetantoine/osirix/). The ratio of tumor SI over muscle SI was used for evaluation of specific tumor enhancement. The region of interest (ROI) was manually drawn from DCE-MRI images (on the left ventricle of the heart for the blood, the whole tumor, the tumor peripheral, the tumor center, or the muscle around the tumor). The SI of ROI from DCE-MRI images was obtained using a home-made MATLAB program.

### Modeling of Contrast Agent Uptake

A linear compartmental model was used to calculate plasma volume (PV) and fractional leakage rate (FLR). The tumor is composed of two compartments: 1) plasma and 2) extravascular and extracellular space.

### Histology Study

Hematoxylin & Eosin stained tumor slices were examined. An estimation of necrosis was made for tumors with a score 1, 2 and 3 to represent 0-33%, 34-65%, and 66-100% necrosis, respectively.

### Statistical Analysis

Statistical analysis was performed using a student t-test (GraphPad Prism; GraphPad Software, San Diego, Calif.). p values were two-tailed with a confidence interval of 95%. The difference was considered significant when p<0.05.

### Characterization of Laser Energy Deposition

The diameter of the laser light spot (y, in cm) is linear to the distance (x, in cm) between the laser fiber tip and the paper: y=0.47 x+0.41. For DyeLA at a distance of 5 mm the light dose applied is 7.7 and 15.4 W/cm² at 2.5 and 5 W, respectively.

As the dye concentration was increased, the temperature of the solution also increased, suggesting the dye helped in absorbing the light energy. Furthermore, higher increases in temperature was induced by higher laser power, indicating that more energy was absorbed at a higher laser power.

### Photothermal Therapies

Tumors usually reach the size of 100 mm³ in about 3 weeks and 500 mm³ in about 5 weeks after tumor cell inoculation. The tumor treated with LA showed a large scale of observable thermal destruction immediately after the treatment. When tumors injected intralesionally with indocyanine green (ICG) were given a laser light dose, changes were obvious: the temperature of tumor increased; the color of the skin overlying the tumor changed to white after the treatment, and brown or black if overheated; the internal explosions could be heard. The injury on the skin surface was inevitable and the degree varied. However, a complete recovery of the skin was usually observed after 1 month. For tumors without ICG injection, no obvious difference was observed during and after laser irradiation.

After treatments, the tumor growth was slowed down for both 500 mm³ and 100 mm³ tumors as presented in Table 3 and Table 4, respectively. For 500 mm³ tumors in Table 3, except for group 2, all treatments were effective to slow down the tumor growth. For DyeLA, the small 100 mm³ tumors group had much smaller tumors than the 500 mm³ tumor group 12 days after treatment: 0.2±0.1 (Table 4) vs. 1.10±0.16 (Table 3). The 100 mm³ tumors group treated by both LA and DyeLA were significantly smaller than the control tumors at both 7 days and 12 days after treatments.

TABLE 3
Normalized tumor size for 500 mm³ tumor groups.

| Group | Therapy | Experiment condition | 7th day | 12th day |
|---|---|---|---|---|
| 1 | None | | 1.54 ± 0.53 | 3.12 ± 1.16 |
| (control) | | | | |
| 2 | LA | 1 W × 10 min, 2 | 1.68 ± 0.45 | N/A |
| | | ablation | | |
| 3 | LA | 2 W × 10 min, 2 | 1.07 ± 0.65 | N/A |
| | | ablation | | |
| 4 | LA | 2 W × 5 min, 4 | 1.05 ± 0.33 | 1.25 ± 0.45* |
| | | ablation | | |
| 5 | LA | 2 W × 5 min, cooling, | 0.69 ± 0.21* | 0.76 ± 0.13* |
| | | 4 ablation | | |
| 6 | DyeLA | 2.5 W × 10 min | 0.79 ± 0.11* | 0.94 ± 0.21* |
| 7 | DyeLA | 5 W × 10 min, cooling | 0.95 ± 0.15 | 1.10 ± 0.16* |

*p < 0.05 when compared to control tumors at the same time (7 days or 12 days).

TABLE 4
Normalized tumor size for 100 mm³ tumor groups.

| | 7 days | 12 days |
|---|---|---|
| Control | 1.6 ± 0.4 | 3.2 ± 0.6 |
| LA | 0.4 ± 0.3* | 0.6 ± 0.4* |
| DyeLA | 0.1 ± 0.1* | 0.2 ± 0.1* |

*p < 0.05 when compared to control tumors at the same time (7 days or 12 days).

The mice were sacrificed 3 weeks after treatment. Mice, which had their tumors completely ablated 3 weeks after treatment, were kept for a total of 3 months to evaluate the long term efficacy. No recurrence of tumors was observed.

### Image Analysis

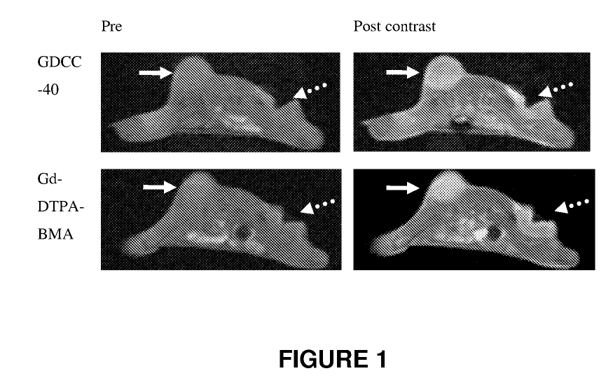

2D SE images of tumors enhanced by Gd-DTPA-BMA or GDCC-40 are shown in FIG. 1. The SI enhancement ratios (SI post/SI pre) of the whole untreated tumor are between Gd-DTPA-BMA (1.60±0.19) and GDCC-40 (1.65±0.21). The residual tumor enhancement ratios are similar between Gd-DTPA-BMA (1.66±0.10) and GDCC-40 (1.51±0.19). In conclusion, the SI enhancement ratios of both the control tumors and the residual tumors are not significantly different between these 2 contrast agents.

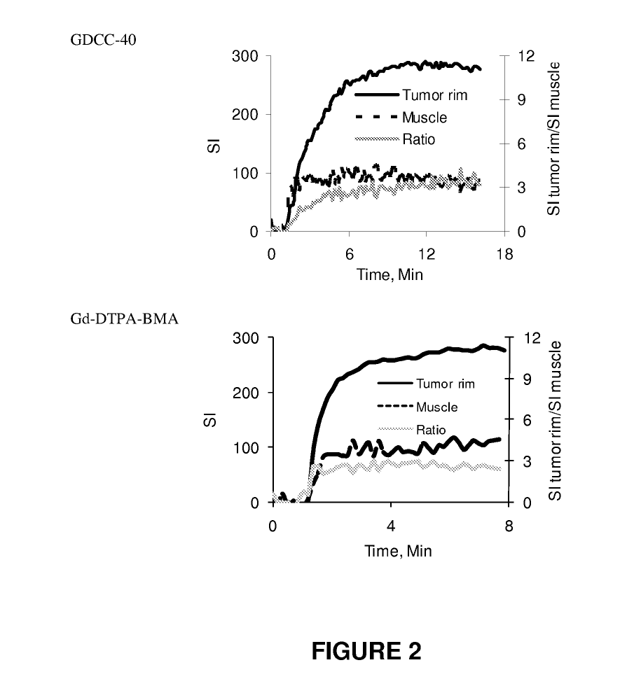

For the specific enhancement of the tumor, the ratio of the SI at the tumor rim to the SI of the muscle was calculated. Sample time courses of the tumor rim and muscle SI ratios are shown in FIG. 2. The SI of muscle reached plateau at about 1.5 min for both GDCC-40 and Gd-DTPA-BMA. Conversely, the SI of tumor rim reached a maximum at about 12 min using GDCC-40 and 7 min using Gd-DTPA-BMA, respectively. The ratio using GDCC-40 kept increasing and reached more than 3 at 15 min As a comparison, the ratio using Gd-DTPA-BMA reached plateau within 2 min and then stayed the same even after 8 min

TABLE 5
PV and FLR for the tumor center, the whole tumor and the tumor rim
enhanced by GDCC-40 and Gd-DTPA-BMA, respectively.

| | GDCC-40 | | | Gd-DTPA-BMA | | |
|---|---|---|---|---|---|---|
| | | Whole | | Tumor | | |
| | Tumor center | tumor | Tumor rim | center | Whole tumor | Tumor rim |
| PV | 0.058 ± 0.046 | 0.076 ± 0.032* | 0.082 ± 0.038* | 0.098 ± 0.019 | 0.138 ± 0.020 | 0.190 ± 0.018 |
| FLR | 3.24 ± 1.10* | 4.57 ± 0.91* | 5.21 ± 0.88* | 9.93 ± 3.19 | 20.86 ± 1.18 | 28.42 ± 4.36 |

*p < 0.05 indicating the difference is significant when comparing GDCC-40 with Gd-DTPA-BMA.

### Histological Analysis

The results showed that slices from the control tumor have a score of 1 with minimal central necrosis. In contrast, those slices from the tumors treated with LA or DyeLA have an average score of 2. Some of these slices have breakages in the peripheral rim of the tumor or have incomplete section of tumor. This finding confirms the efficacy of photothermal therapies to tumor.

### Discussion

For 500 mm³ tumor (Table 3), all groups except group 2 were effective at slowing down tumor growth, which indicates that the damage by 1 W laser power was not sufficient to slow down the growth of the whole tumor. 12 days after treatment, groups 4 to 7 showed significantly slower growth rates. However, none of the treatments could eradicate the tumor completely and the tumors kept growing 12 days after treatment.

It was speculated that the efficacy of the treatments was hindered by the large size of the tumor (500 mm³) The efficacy was dramatically improved for the 100 mm³ tumor group (Table 4). The tumor size shrank 7 days after treatment, with an average size of 0.4 and 0.1 observed for LA and DyeLA, respectively. However, the problem of an incomplete ablation still existed for LA even after treatment with multiple ablations. No single tumor treated by LA reached complete ablation and the tumor residual kept growing after treatment.

For LA, 2 W proved to be effective in slowing down the tumor growth and it was easy to control. A power of 5 W was tried but it was too strong, causing damage to the surrounding healthy tissue. The bare laser fiber tip used in this study created a very poor radial temperature profile in the tumor. The highest temperature is at the tip of fiber and drops quickly as the distance increases. Thus overheating always burns tissue adjacent to the laser tip while the tissue even at several mm away is not affected. Although multiple ablations were used, complete eradication of the tumor was rarely achieved. The place with the most recurrence of tumor was the interior side of the tumor peripheral rim, which is the region of the tumor the laser fiber has difficulty covering. A commercial cooled power laser system for LA has 2 features to optimize the efficacy: 1) a diffuser or applicator in front of the fiber tip to scatter the laser light and therefore maximize the irradiation range, and 2) the internal cooling system to shift the maximum temperature deeper into the tumor and thus avoid burning the surrounding tissue.

Compared to LA, DyeLA was less technically demanding for treatment planning and execution, especially for the 100 mm³ tumor group. 7 out of 10 tumors were completely eradicated 7 days after treatment. There was no recurrent tumor growth 12 days after treatment and local site had been tumor free 60 days after the treatment so far. At the treatment site, the skin was recovered. After the skin was opened, there was no sign of burning on the muscle underneath the treatment site. The tumor residual was possibly attributed to possibly uneven ICG distribution and/or uneven laser energy application.

For the control and residual tumors, 2D MR images showed similar SI enhancement using either Gd-DTPA-BMA or GDCC-40. The GDCC-40 enhancement of the tumor started slow, but reached similar contrast (SI of tumor rim to SI of muscle) compared to Gd-DTPA-BMA about 4 min after injection. The linear compartmental model was used to analyze the early stage of contrast agent wash-in, assuming the reflux from the tumor to the vasculature is ignorable at the early stage of perfusion.

The PV values (Table 5) estimated using GDCC-40 were very reasonable (0.058 to 0.082) compared to those estimated using Albumin-Gd-DTPA (0.023 to 0.065) (36). In contrast, those measured using Gd-DTPA-BMA (0.098 to 0.190) seem too high.

The estimated FLR values for GDCC-40 (Table 5, 3.24 to 5.21 1/hr) were again more accurate than GD-DTPA-BMA (14.93 to 28.42 1/hr) when compared to Albumin-Gd-DTPA (1.27 to 8.10 1/hr) (36).

Therefore, to monitor the efficacy of an anti-cancer treatment, GDCC-40 and Gd-DTPA-BMA exhibit similar enhancement and contrast in static MR images. However, regarding the tumor parameters from DCE-MRI such as PV and FLR, estimations from GDCC-40 are more reasonable compared to those of Gd-DTPA-BMA. Furthermore, only several minutes are needed for accurate estimation of permeability using GDCC-40. During this time period, even with some degradation, the size of GDCC-40 is relatively big to differentiate the leakiness of the vasculature. This again proves that a small molecular weight contrast agent such as Gd-DTPA-BMA is not a good choice to differentiate the vasculature leakiness.

In summary, DyeLA is an effective anti-cancer treatment for small tumors. A complete cure could be accomplished without damages to non-selective healthy tissue. GDCC-40 is a suitable contrast agent for MRI in diagnosing tumors and assessing efficacy of anti-cancer therapy non-invasively.

### II. Pharmacodynamics of GDCC-40 for DCE-MRI and the Correlation of Acute Tumor Response and Residual Tumor

### Materials and Methods

Same as those described above.

### Animal Tumor Model

Female athymic nude mice (5-6 weeks old, Frederick, M D, National Cancer Institute) were cared for under the guidelines of a protocol approved by the University of Utah Institutional Animal Care and Use Committee. The MDA-MB-231 human breast cancer cell line was purchased from the American Type Culture Collection (ATCC, Manassas, Va.) and cultured in Leibovitz's L-15 medium with 2 mM L-glutamine and 10% FBS at 37° C. in a humidified atmosphere of 5% CO₂. 5×10⁶ cells in a mixture of 50 μL medium and 50 μL Matrigel (Becton-Dikinson, Franklin Lakes, N.J.) were inoculated subcutaneously on both sides of the hips of each mouse. Each implantation generated a tumor. Tumors about 10 mm³ were used for interstitial laser ablation (LA) and dye enhanced LA (DyeLA). After the treatment each mouse was kept in individual cage to avoid the chewing of tumor by other mice.

### Laser Ablation

DenLaser 800 (Cao Group Inc., Salt Lake City, Utah) was used for both LA and ICG enhanced photothermal therapy (DyeLA). This Diode laser operates at λ=810 nm with the aiming beam at λ=650 nm. For LA, the trajectory of the laser fiber was created by a puncture of a 25 G needle. Laser ablations were applied 4 times: the first two along the longest axis of the tumor in parallel about 3 mm apart and then the second one perpendicular to the first one also in parallel about 3 mm apart, giving a total of 4 ablations for each tumor. The application time of the laser was evenly distributed among all ablations.

Details of the parameters for DyeLA are shown in Table 6. 1.5% DI water solution of indocyanine green was prepared and 30 uL were injected intra-tumorally 4 hr before the treatment. The laser tip was kept 5 mm away from the skin. Dripping water was used to cool down skin temperature.

The size of the tumors were monitored regularly. Size=π/6×D1×D2×D3 (π=3.14, D1, D2, and D3 are the tumor length, width, and thickness measured by digital caliber, respectively). Each tumor size was normalized to the size at the treatment day (Day 0): ratio=Size_{Day 7 or day 12}/Size_{Day 0.}

TABLE 6
Parameters for treatment of breast tumors (100 mm³) in nude mice.

| Group | Light dose | Cooling | Light dose | No. of mice |
|---|---|---|---|---|
| Control | 0 | 0 | 0 | 6 |
| LA | 2 W; 6 min | Yes | 720 J | 6 |
| DyeLA | 5 W; 12 min | Yes | 11.1 KJ/cm² | 10 |

### MRI Scan Procedure

Tumor bearing mice were subjected to MR scans using a Siemens Trio 3T scanner. The mice were anesthetized by the intraperitoneal administration of a mixture of ketamine (90 mg/kg) and xylazine (10 mg/kg). 4 hr and 7 days after treatment, GDCC-40 was injected intraveneously via tail vein cannulization at a dose of 0.05 mmol-Gd/kg mouse body weight. Lower doses of 0.01 and 0.025 mmol-Gd/kg mouse body weight were used 9 days after treatment.

2D fast low angle shot pulse sequence (2D FLASH) was used for DCE-MRI with the following parameters: TR/TE=104/4.46 ms, α=30, 0.5×0.5×1.5 mm, average=1, acquisition time=11 sec, and a total 10 slices per scan. The system body coil was used for RF excitation and a human wrist coil was used for RF reception. 2D FLASH was repetitively acquired for 16 min Contrast agent was injected 45 sec after the 2D FLASH started to acquire 4 scans for the calculation of baseline signal intensity (SI).

2D spin echo (SE) images were acquired before contrast agent injection and at the end of 2D FLASH with the following parameters: TR/TE=400/10 ms, α=90, 0.4×0.4×2 mm, average=2, acquisition time=61 sec.

### Image Analysis

SI of the untreated whole tumor pre and post contrast enhancement in 2D SE images was obtained using Osirix (http://homepage.mac.com/rossetantoine/osirix/). The ratio of tumor SI over muscle SI was used for evaluation of specific tumor enhancement. The region of interest (ROI) was manually drawn on DCE-MRI images: on the left ventricle of the heart for the blood and the whole tumor, The SI of ROI from DCE-MRI images was obtained using a home-made MATLAB program.

### Modeling of Contrast Agent Uptake

A linear compartmental model was used to calculate plasma volume (PV), fractional leakage rate (FLR), and permeability surface area product (PS) (Shames D M, Kuwatsuru R, Vexler V, Muhler A, Brasch R C. “Measurement of capillary permeability to macromolecules by dynamic magnetic resonance imaging: a quantitative noninvasive technique” Magn Reson Med 1993; 29:616-622).

### Statistical Analysis

Statistical analysis was performed using the student t test (GraphPad Prism; GraphPad Software, San Diego, Calif.). p values were two-tailed with a confidence interval of 95%. A difference was considered significant when p<0.05.

### Laser Ablation

Both LA and DyeLA are effective anti-cancer treatments. The growth rates of the treated tumors were significantly slower than those of the control tumors measured as shown in Table 7.

TABLE 7
Tumor size at 7 days and 12 days after treatment.

| | 7 days | 12 days |
|---|---|---|
| Control | 1.6 ± 0.4 | 3.2 ± 0.6 |
| LA | 0.4 ± 0.3* | 0.6 ± 0.4* |
| DyeLA | 0.1 ± 0.1* | 0.3 ± 0.5* |

*p < 0.05 indicating that the difference between treated and control tumors is significant.

### Image Analysis

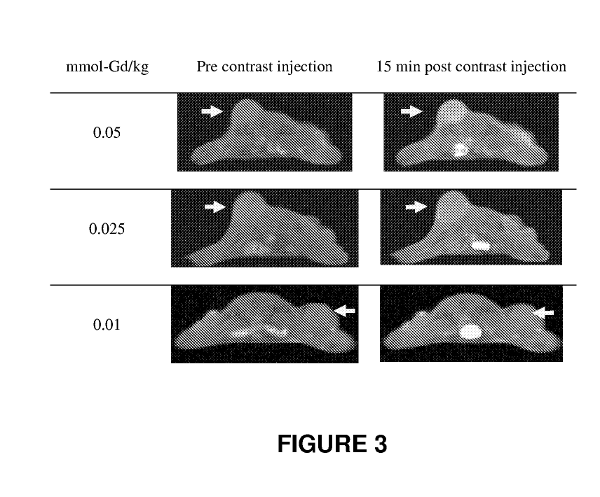

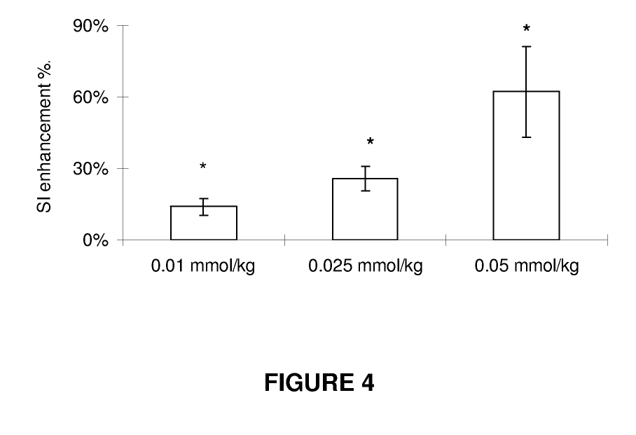

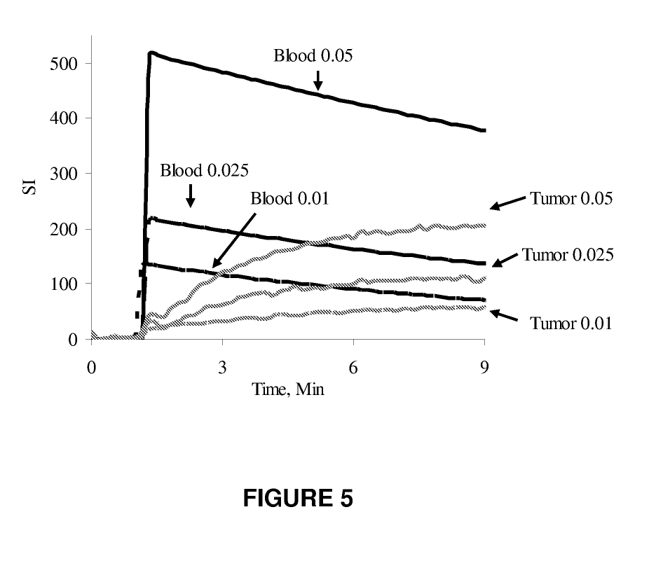

2D SE images enhanced by various doses of GDCC-40 at least 7 days after treatment are shown in FIG. 3. There was still visible tumor enhancement even at the lowest dose (0.01 mmol-Gd/kg). The untreated tumor enhancement by GDCC-40 are 0.14±0.03, 0.26±0.05, and 0.62±0.19 for 0.01, 0.025, and 0.05 mmol-Gd/kg, respectively, as shown in FIG. 4. Representative signal intensity time courses for blood and untreated tumor at 0.01, 0.025, and 0.05 mmol-Gd/kg are shown in FIG. 5. The magnitude of the curves is roughly proportional to the contrast agent dose for both blood SI and tumor SI.

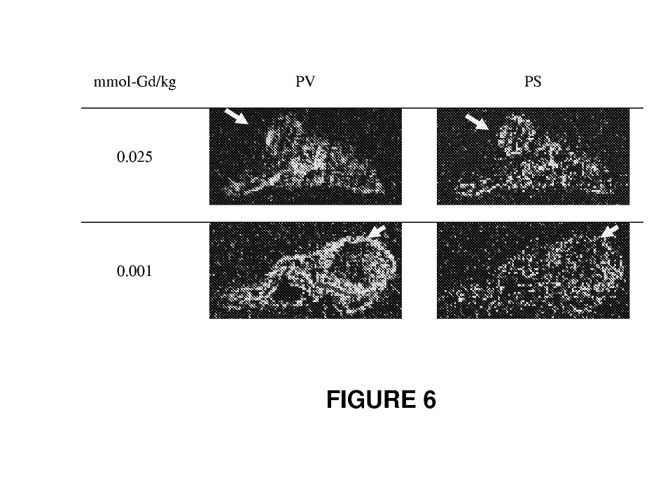

PS and PV mapping of tumor bearing mice using different doses are presented in FIG. 6. PV and PS maps clearly show the location of the tumor and its heterogeneity. The comparison of PV and PS enhanced by 0.01, 0.025 and 0.05 mmol-Gd/kg GDCC-40 for the untreated tumor are listed in Table 8.

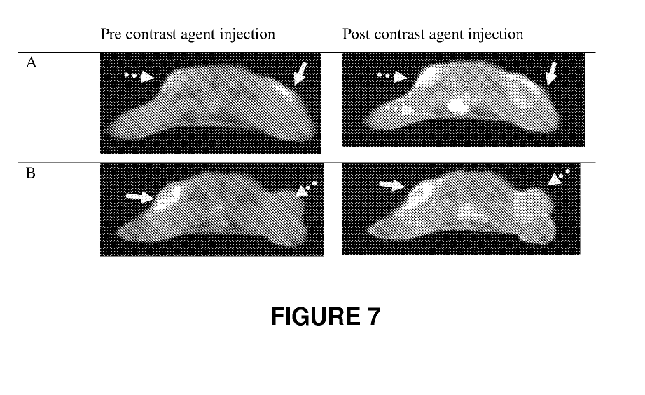

2D SE images for acute response at 4 hr after LA before and 15 min after injection of 0.05 mmol-Gd/kg GDCC-40 are shown in FIG. 7.

TABLE 8
Values of PV, FLR, and PS for the untreated tumor enhanced by 0.01,
0.025, and 0.05 mmol-Gd/kg GDCC-40.

| | 0.01 | 0.025 | 0.05 |
|---|---|---|---|
| PV | 0.124 ± 0.045 | 0.123 ± 0.059 | 0.103 ± 0.043 |
| FLR | 6.05 ± 3.58 | 5.06 ± 1.84 | 3.95 ± 0.81 |
| PS | 0.997 ± 0.613 | 0.611 ± 0.351 | 0.517 ± 0.216 |

Inflammation was noticeable in the images before contrast agent injection as shown in FIG. 7. The bright area seen in the pre and post contrast agent injection images is from the inflammation caused by thermal damage of LA and DyeLA (pointed out by the solid arrows in FIG. 7). Treated tumor exhibited damaged vasculature, which had less uptake of contrast agent and, therefore, less MR enhancement.

The damage along the trajectories of LA was visible (FIG. 7B). The tissue around the fiber tip was burned, resulting in black holes. The damage from DyeLA was more evenly distributed within the tumor. The inflammation was close to the skin overlying the tumor where the laser irradiated.

In post contrast agent images of the untreated tumor, the ratio of tumor SI over muscle SI was 1.50±0.05. For treated tumor, the ratios were 1.29±0.03 for DyeLA and 1.42±0.33 for LA. Furthermore, much higher standard deviation was observed for LA, indicating the heterogeneous nature of LA ablation.

### DCE-MRI Image Analysis and Modeling

The values of PV, FLR, PS, and their coefficient of variation for LA, DyeLA, and untreated tumors are compared in Table 9. Due to the heterogeneity of these treatments, the coefficient of variation (CV) of all three parameters (PV, FLR and PS) of untreated tumors was smaller than those of treated tumors (CV_{untreated}/CV_{LA} and CV_{untreated}/CV_{DyeLA} in Table 9).

TABLE 9
Comparison of PV, FLR, PS, and their coefficient of variation for LA,
DyeLA, and untreated tumors.

| | | | | CV_{untreated}/ | CV_{untreated}/ |
|---|---|---|---|---|---|
| | LA | DyeLA | Untreated | CV_{LA} | CV_{DyeLA} |
| PV | 0.028 ± 0.020 | 0.038 ± 0.021 | 0.103 ± 0.043* | 0.27 | 0.37 |
| FLR | 1.63 ± 0.51 | 1.89 ± 1.05 | 3.95 ± 0.81* | 0.41 | 0.48 |
| PS | 0.090 ± 0.042 | 0.138 ± 0.140 | 0.517 ± 0.216* | 0.17 | 0.27 |

*p < 0.05 between untreated tumors and treated tumors (both LA and DyeLA)

### Correlation Between Acute Response and Residual Tumors

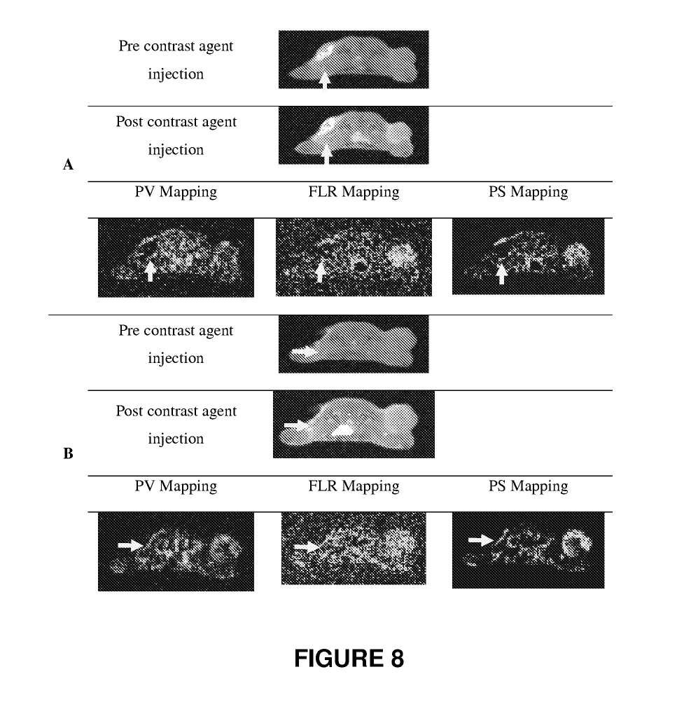

One tumor treated with LA was scanned 4 hr and 7 days after treatment. The 2D SE images and mapping of PV, FLR and PS are depicted in FIG. 8. There is a good correlation between acute response and the residual tumor. The arrows point to the suspicious area that is overshadowed by the bright inflammation but has hyper permeability. 7 days after treatment, the same area shows strong contrast agent uptake, proving it is a residual tumor.

### Discussion

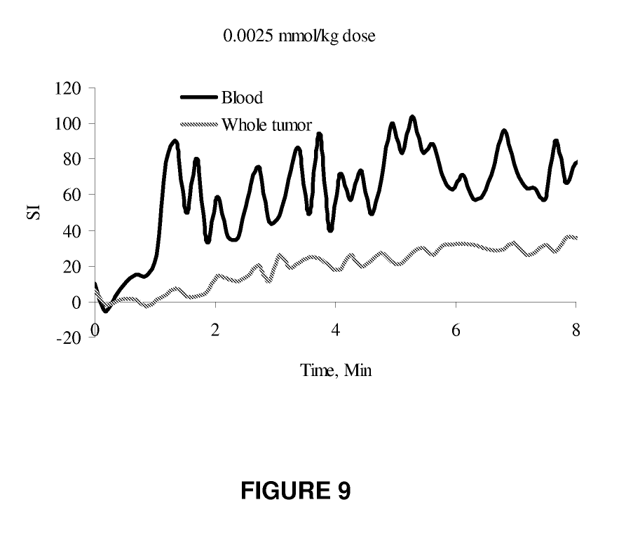

The parameters estimated using 0.01 and 0.025 mmol-Gd/kg doses of GDCC-40 for untreated tumor imaging were close to those using 0.05 mmol-Gd/kg: 0.124, 0.123, and 0.076 for PV; 6.05, 5.06, and 4.57 for FLR estimated by 0.01, 0.025 and 0.05 mmol/kg GDCC-40, respectively. Since lower SI of tumor was obtained from MR images, image analysis at lower doses demanded more careful ROI selection of tumor. Signal intensity for blood with 0.0025 mmol-Gd/kg was tested once. The SI of tumor and heart using 0.0025 mmol-Gd/kg GDCC-40 is shown in FIG. 9. The initial infusion of GDCC-40 into tumor blood was hard to differentiate from later uptake by the leaky vessel. Therefore GDCC-40 at 0.05 mmol/kg is a better choice compared to lower doses. Performance of lower doses could be improved by using a more specific coil such as smaller diameter rat coil and higher magnet field such as the 7T animal MRI scanner to improve signal to noisy ratio.

Acute response 4 hr after treatment using GDCC-40 enhanced MRI was able to reveal the location and severity of the damage. Two thermal therapies based on different principles showed different characteristics of the damage. LA using bare laser fiber caused burning only in the vicinity, while DyeLA caused damage along the path laser light traveled through. For DyeLA, the orientation of laser fiber and the light dose are critical to the success of treatment, therefore, MRI should be incorporated at the diagnosis stage to provide spatial information about the tumor and facilitate treatment design.

Heterogonous response of the tumor was observed such as high signal intensity region on the side of the tumor where light irradiated, representing the prompt edema and inflammatory infiltration at the dermis of the skin, which were common features after thermotherapy. The depth of the necrosis measured histologically could be correlated very well with that using MR images.

It is not easy to have ICG distributed evenly inside the tumor after injection. Only one puncture in the tumor is allowed during the injection of ICG. Even part of the tumor has no ICG, no extra ICG could be injected via the second puncture since ICG will leak out though the first one. That part of the tumor tissue has no ICG or very little ICG will likely survive the treatment depending on its location and light dose. One minor side-effect is that the healthy tissue surrounding tumor could get damaged if there is ICG in the tissue.

10% of a clinically approved dose of GDCC-40 (0.01 mmol-Gd/kg mouse body weight) was tested and the results were similar to those of 0.05 mmol-Gd/kg dose. Using a lower dose resulted in a lower accumulation of toxic Gd (III), making it safer to use. Increased dosages lead to easier image analysis and improved accuracy. Acute response assessed by photothermotherapy DCE-MRI using GDCC-40 is valuable in timely and accurately evaluating anti-cancer treatments as well as for prognosis purposes.

### III. In Vivo DEC-MRI Assessment of Anti-Angiogenic Effect of Avastin in a Colon Carcinoma Model Using GDCC

### Materials and Methods

Tumor and Animal models

The study protocol was approved by the local ethics committee for animal care and use. HT-29 human colon carcinoma cell line was purchased from American Type Culture Collection (ATCC, Manassas, Va.) with ATCC number HTB-38™. HT-29 cell line was cultured using ATCC complete growth medium (5 McCoy's Medium with 10% fetal bovine serum).

Athymic female NCr-nu/nu nude mice at 4 weeks were purchased form National Cancer Institute at Frederick, M D. Concentrated HT-29 cells in complete medium was mixed with Matrigel basement membrane matrix (BD Biosciences, San Jose, Calif.) at 1:1 ratio. 2×10⁶ cells in 100 μl mixture were implanted s.c. in the flank of 12 mice weighing about 23 g.

### Contrast Agents

Gd-DTPA cystamine copolymers (GDCC40K, MW: 40 KDa) were prepared as described above. They were further fractionated using a Sephacryl S-300 column on a Pharmacia FPLC system (Gaithersburg, Md.) to prepare the agents with narrow molecular weight distributions. The average molecular weights of the fractions were determined by size exclusion chromatography using poly[N-(2-hydroxypropyl)methacrylamide] as standard on an AKTA FPLC system (G E Biosciences, Piscataway, N.J.). The Gd(III) content in the agents was determined by inductively coupled plasma optical emission spectroscopy (ICP-OES, Perkin Elmer Optima 3100XL). Omniscan® (Gd-DTPA-BMA, gadodiamide, MW: 574 Da) was obtained from Nycomed Inc., Princeton, N.J.

### MRI Protocol

Mice were anesthetized with an intraperitoneal injection of a mixture of ketamine (Bedford, Ohio, 1000 mg/kg) and xylazine (St. Joseph, Mo., 10 mg/kg). They were then placed supine with the tumors located approximately at the center of a human wrist coil. A tail vein of mouse was catheterized using 30 gauge needle connected with heparinized saline filled 2.5 m long tube. About 100 μL of contrast agent was injected via the tubing and 200 μL saline was used to flush the tubing after contrast agent injection. The dose for Omniscan is 0.1 mmol-Gd/kg, for GDCC40K is 0.05 mmol-Gd/kg. All images were acquired on a Siemens Trio 3T scanner using a system body coil for RF excitation and a human wrist coil for RF reception. A group of 4-6 mice weighing 23 grams was used for each agent.

Before contrast agent injection, one 3D fast low angle shot (FLASH) image and 2D coronal spin echo (SE) images were acquired. The 3D FLASH image was used to define the regions of interest for 2D SE image. The coronal slices in 2D SE images were selected for the acquisition of DCE-MRI data. Dynamic MRI scan was performed using 2D FLASH for a period of 20 min After a 1-2 min delay, the contrast agent was administered via the tubing. 2D coronal SE scan was acquired at 20 min after the injection. Parameters of the 3D FLASH pulse sequence are: TR/TE=7.40/2.60 ms, α=25°, 0.5 mm×0.5 mm×0.5 mm coronal slice thickness. Parameters of the 2D SE pulse sequence are: TR/TE=600/13 ms, α=90°, 0.4 mm×0.4 mm×0.8 mm axial slice thickness. The parameters of 2D FLASH pulse sequences for DCE-MRI scan are: TR/TE=5.80/2.10 ms, 25 RF flip angle, 0.8 mm×0.8 mm×0.8 mm axial slice thickness, single acquisition, total 20 axial slices (several slices cover the heart and the rest cover majority of the tumor), and scan time is 6 (or 5.4) seconds for a single acquisition.

### Experimental Protocol

Animals were selected for study when the tumors reached approximately 1 cm in diameter (about 3 weeks after tumor cells inoculation) and were randomly divided into two groups (each group n=6, group1 and group2). One group (group1) underwent DCE-MRI with macromolecular contrast agent-GDCC40K (MW: 40 KDa), and another group (group2) underwent DCE-MRI with small molecular weight contrast agent—Ominiscan (MW: 574 Da). Before treatment, Ominiscan was injected at 0.1 mmol-Gd/kg for group1, GDCC40OK (40K Da) was injected at 0.05 mmol-Gd/kg for group2. After the baseline MRI examinations, both groups were intraperitoneally (i. p.) injected Avastin at a dose of 200 μg/mouse (0.1 ml) every 2 days for 1 week. 36 h and 7 days after initiating Avastin treatment, all of the animals were imaged for second and third time by the same protocol applied for the baseline MR examinations.

In addition, tumor size of all animals was determined using a caliper during the study.

### Quantitative Analysis of DCE-MRI Data

MR imaging data were analyzed by using a general kinetic two-compartment bidirectional exchange model as shown in equation 1 in the Matlab programming environment (The MathWorks, Inc., Natick, Mass.).

$${C}_{T} ( t ) = {K}^{trans} {\int }_{0}^{t} {C}_{p} ( θ ) {e}^{- {k}_{ep}} d θ + {f}_{PV} {C}_{p} ( t ) \qquad ( 1 )$$

where Cₚ is the contrast agent concentration in the plasma space, C_{T} is the contrast agent concentration in the tumor, K^{trans} is the endothelial transfer coefficient and kₑₚ is the rate constant of reflux from the extravascular and extracellular space (EES) back to plasma, θ is Laplace operator, f_{PV} is the vascular volume fraction. In order to estimate K^{trans} and f_{PV}, the Eq. 1 was fitted to the experimental signal intensity values of the heart and tumor rim using a nonlinear algorithm.

Images were analyzed on the selected regions of interest (ROIs) to obtain the average values of K^{trans} and f_{PV} or on a pixel-by-pixel basis to obtain parameter maps of K^{trans} and f_{PV}. ROIs were manually tracked to cover the tumor rim. Quantitative kinetic analysis of tumor enhancement was limited to the tumor rim due to the most vascularized tumor periphery and the scarce penetration of the contrast agent in the tumor core. To obtain signal intensities (SI) of the blood, the right ventricle of heart was selected as ROIs. The average MR signal intensity of ROI before the contrast agent injection (SI₀) was used as the baseline. Signal changes after contrast injection were calculated as: ΔSIₜ=(SIₜ−SI₀)/SI₀, where SIₜ is the signal intensity within the ROI at the time t of post-contrast. The signal intensity modeled here is dependent on the pre-contrast T1 of the ROI. Variations in the native tissue T1 values will affect the measured signal intensity; however, since T1 values of benign and malignant lesions show considerable overlap, and moreover the same tumor tissue was compared every time in the current study, T1 values should have not significant variation for the same tissue before and after drug treatment. Thus, the results here may not be strongly affected and it can be assumed that ΔSI is proportional to the change of the contrast agent concentration, which is a reasonable approximation at low contrast agent concentration.

### Statistics Analysis

K^{trans} and f_{PV} from the same tumor before and after therapy (treatment or control) for each contrast agent were compared. Statistical analysis was performed using a paired two-tailed Student's t-test (GraphPad Prism; GraphPad Software, San Diego, Calif.). A confidence interval 95% (P<0.05) was considered statistically significant.

### Results

### Effect of Avastin on Growth of HT-29 Colon Cancer

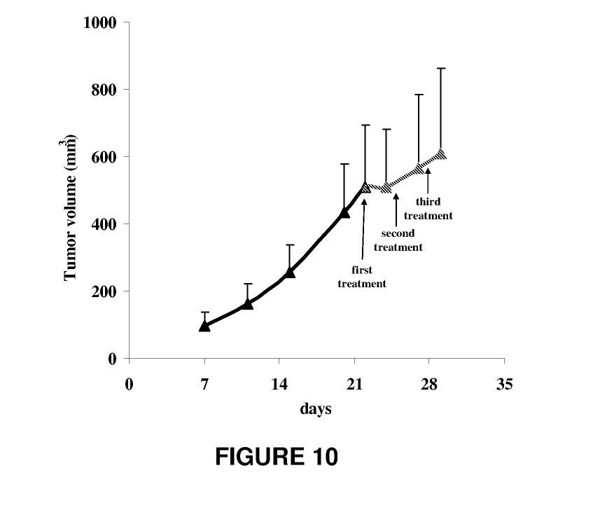

The tumors successfully grew in all implanted mice. 3 weeks after inoculation (the time of baseline MRI examinations for pretreated mice), the mean size of all the tumors (from both groups) reached about 508±185 mm³. 36 h after Avastin-treatment, the tumor growth was largely inhibited and the tumor size kept almost the same as pre-treatment (510±171 mm³) Following the second and third Avastin-treatment, the tumors grew gradually again. However, the tumor growth became much slower than that of pre-treatment (FIG. 10).

### Dynamic Contrast Enhanced MR Images

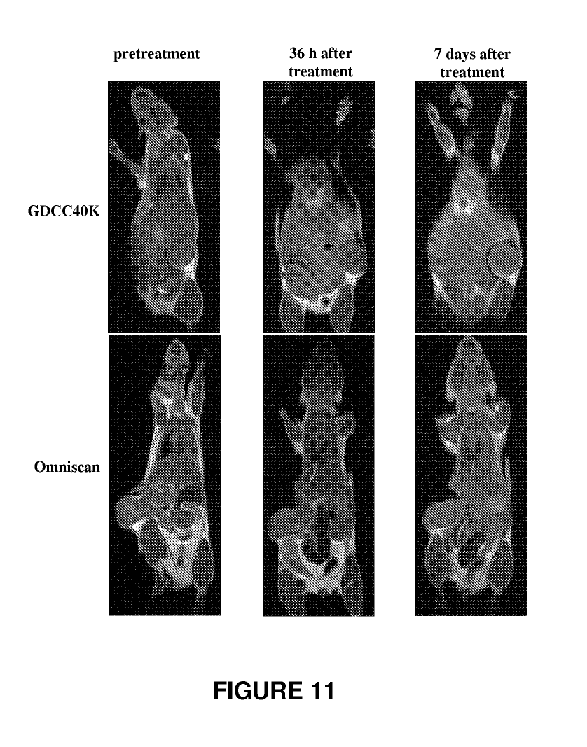

FIG. 11 shows the representative 2D coronal spin echo (SE) images acquired 20 minutes after injection of a bolus of contrast agents in two randomly chosen tumors from GDCC40K and Omniscan injection groups, respectively. The signal intensity enhancement was more pronounced in the peripheral region of tumor, which has been proved to be the region most representative of angiogenic activity. Thus, tumor rim was selected as ROIs for analysis. Signal intensity from heart was used as the plasma data. However, change in contrast enhancement before and after drug treatment is difficult to assess visually. In order to get more information from the DCE-MRI data, signal intensity-vs-time dynamic curves of tumor were calculated and compared next.

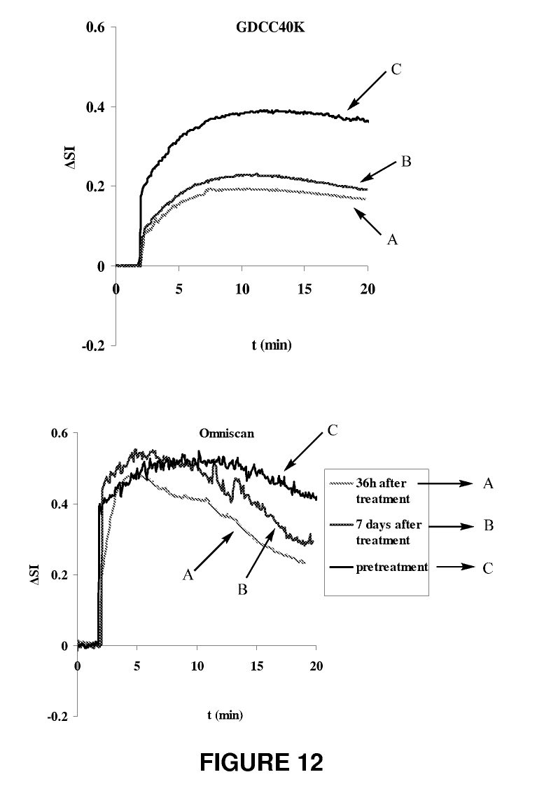

FIG. 12 shows some representative graphs of contrast enhancement-time curves for GDCC40K and Omniscan before, 36 h and 7 days after administration of Avastin. For both GDCC40K and Omniscan, the contrast enhancement of tumor tissue decreased after drug treatment.

K^{trans} and f_{PV} values were used to evaluate the effects of Avastin on permeability and vascular volume fraction, respectively, of the tumor microvasculature. The mean values for K^{trans} and f_{PV} obtained from GDCC40K and Omniscan before and after drug treatment were summarized in Table 10. Interestingly, the contrast enhancement of tumor 36 h after single dose treatment decreased more prominently than that of 7 days after multi-dose treatment. From these results, it seemed that the single dose treatment was more effective than multi-dose treatment.

TABLE 10
Comparison of calculated DCE-MRI derived parameters before and after
administration of Avastin.

| | | K^{trans} | | | fₚᵥ | | |
|---|---|---|---|---|---|---|---|
| | | | | 7 days after | | | 7 days after |
| | | pretreatment | 36 h after treatment | treatment | pretreatment | 36 h after treatment | treatment |
| GDCC40K | Mean ± SD | 0.014 ± 0.002 | 0.008 ± 0.004 | 0.028 ± 0.029 | 0.028 ± 0.005 | 0.008 ± 0.005 | 0.030 ± 0.022 |
| | P value | | 0.036* | 0.477 | | 0.015* | 0.939 |
| Omniscan | Mean ± SD | 0.07 ± 0.013 | 0.066 ± 0.047 | 0.069 ± 0.055 | 0.102 ± 0.047 | 0.062 ± 0.039 | 0.137 ± 0.034 |
| | P value | | 0.815 | 0.936 | | 0.214 | 0.970 |

K^{trans} and f_{PV} Values for GDCC40K

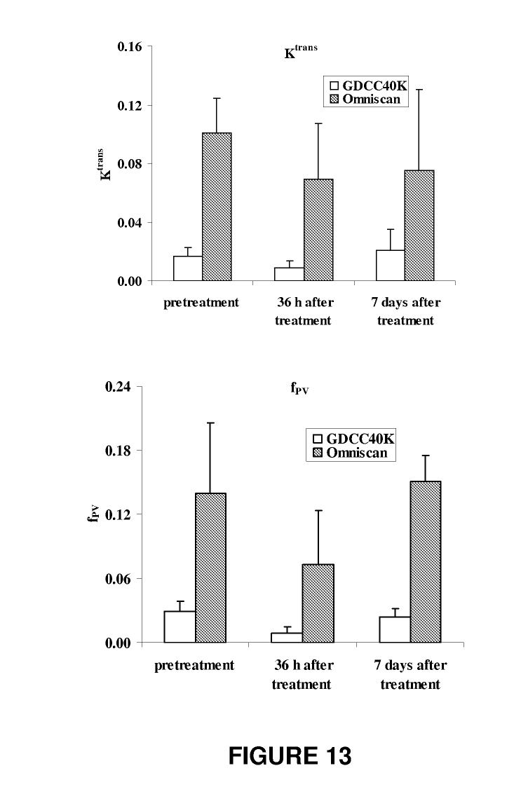

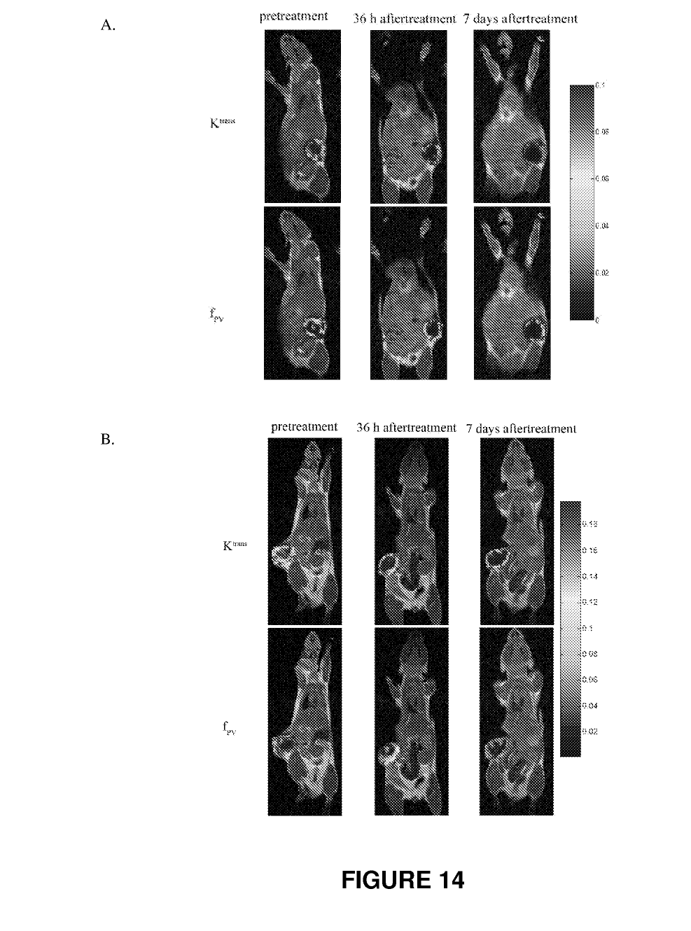

Both K^{trans} and f_{PV} for GDCC40K were significantly decreased at 36 h (P<0.05 for both) after Avastin treatment. However, there was no significantly difference for both K^{trans} (P=0.477) and f_{PV} (P=0.939) at 7 days after Avastin administration compared to pretreatment (FIG. 13).

K^{trans} and f_{PV} Values for Omniscan

Using small molecular contrast agent, Omniscan, mean K^{trans} and f_{PV} values between pre- and post-treatment (including 36 h and 7 days after treatment) exam did not reach any statistical significance (P>0.05), though K^{trans} 36 h after treatment showed decrease compared to pretreatment (FIG. 13).

Color Maps for K^{trans} and f_{PV}

Visualization of parameters is very useful to get intuitionistic information. FIG. 14 showed representative color maps of pretreatment, 36 h and 7 days post-treatment distribution of tumor K^{trans} and f_{PV} values in two mice (one from GDCC40K injection group, one from Omniscan injection group). For GDCC40K injection group, the tumor showed voxels with relatively low K^{trans} and f_{PV} values. After 36 h and 7 days, obvious decreases in voxels with K^{trans} and f_{PV} values were observed compared to pre-treatment (FIG. 14A). For Omniscan injection group, the tumor showed voxels with relatively high K^{trans} and f_{PV} values, however, there was no obvious difference in voxels with K^{trans} or f_{PV} values between pre- and post-treatment (FIG. 14B).

### Discussion

The activity of Avastin, an anti-VEGF antibody in an experimental tumor, was evaluated on HT-29 human colon cancer cell using DCE-MRI. At the early stage (36 h after a single dose administration of Avastin), dynamic MRI enhanced with GDCC40K showed significant declines both in tumor blood vessel permeability, K^{trans}, and vascular volume fraction, f_{PV}. 7 days after 3 doses administration of Avastin, however, K^{trans} and f_{PV} returned back and there was no significant difference compared to baseline before treatment with anti-VEGF antibody. Meanwhile, a decline of K^{trans} was observed in two of 3. Conversely, one of 3 demonstrated an increase in K^{trans}. Though f_{PV} of all the three mice declined, the changes were not significant.

Dynamic MRI enhanced with Omniscan, a small molecular contrast agent, did not show any significant decline in K^{trans} or f_{PV} after single- or multi-dose treatment with Avastin. This result also suggests that small molecular contrast agent may be not a good choice to characterize tumor.

From the results of DCE-MRI with both GDCC40K and Omniscan, it seemed that early therapy effect of a single dose treatment was more effective than multi-dose treatment for 7 days. This coincided with the results of tumor growth which was shown in FIG. 10. 36 h after Avastin-treatment with a single dose, the tumor growth was largely inhibited and the tumor size kept almost the same as pre-treatment. Following the second and third dose of Avastin-treatment, the tumors grew gradually again. However, the tumor growth became much slower than that of pre-treatment (FIG. 10). This result is similar to several other reports that showed treatment with various anti-tumor agents was associated with a significant reduction in tumor growth rate, but the tumor growth can not be completely stopped or reversed.

Usually, multi-dose treatment should be more effective than single dose treatment. Interestingly, an “opposite” result was observed in the current study. There are several explanations for this “opposite” result. First, 50-100 μg/mouse of Avastin was enough to completely inhibit human VEGF-A and the terminal half-life of Avastin is 1-2 weeks in mice body. In this study, 200 ug/mouse Avastin was used. Thus, the single dose treatment was enough and could keep long enough to completely inhibit human VEGF-A during this study. Therefore, multi-dose treatment could not give more contribution to inhibit human VEGF-A. Secondly, as an anti-VEGF monoclonal antibody, Avastin can bind to and neutralize all human VEGF-A isoforms, but not mouse or rat VEGF. In this study, human tumor was implanted in mouse body. Therefore, after Avastin neutralizing the human VEGF-A, a compensatory up-regulated murine VEGF released by host cells and infiltrated into the human tumor cells. The murine VEGF is probably responsible for the tumor angiogenesis and growth. Furthermore, Avastin does not neutralize other members of the VEGF gene family, such as VEGF-B or VEGF-C, or cytokines such as basic fibroblastic growth factor (bFGF), platelet-derived growth factor (PDGF), and epidermal growth factor (EGF), which may also play important roles in stimulating tumor growth. After the human VEGF-A is inhibited by Avastin, these factors may be responsible for the further tumor growth by a compensatory agonist mechanism.

In summary, DCE-MRI with macromolecular GDCC40K can monitor the therapeutic effects of an anti-VEGF antibody on tumor microvessels. Thus, biodegradable macromolecular contrast agents may provide a strong clinical implementation to evaluate and monitor tumor due to their degradability in vivo.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

1. A method for evaluating the treatment of a tumor present in a subject, the method comprising:
a. administering to the subject a contrast agent, wherein the contrast agent comprises a biodegradable macromolecular Gd(III) complex;
b. evaluating a tumor property by magnetic resonance imaging to establish a baseline of the tumor property;
c. treating the tumor in the subject;
d. evaluating the tumor property after step (c) by magnetic resonance imaging; and
e. comparing the results of step (d) with the baseline in step (b) to determine the effectiveness of the tumor treatment.

2. The method of claim 1, wherein the macromolecular Gd(III) complex comprises the formula I 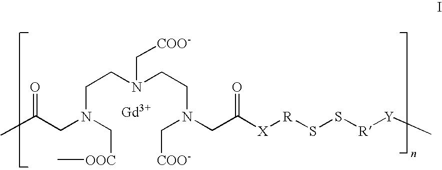
wherein R and R′ comprises, independently, a C₁ to C₁₈ alkyl group, a substituted alkyl group, an unsubstituted aryl group or an aryl group substituted with one or more functional groups comprising an alkyl group, an aryl group, a polyethylene glycol, a saccharide, an amino acid, a peptide, a protein, a peptide conjugate, or a protein conjugate;
X and Y are, independently, O and NH; and
n is an integer between 2 and 10,000.

3. The method of claim 1, wherein the macromolecular Gd(III) complex comprises the formula II 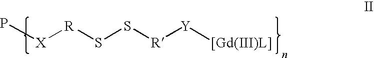
wherein R and R′ comprises, independently, a C₁ to C₁₈ alkyl group, a substituted alkyl group, an unsubstituted aryl group or an aryl group substituted with one or more functional groups comprising an alkyl group, an aryl group, a polyethylene glycol, a saccharide, an amino acid, a peptide, a protein, a peptide conjugate, or a protein conjugate;
X and Y comprises, independently, an amide group, an ester group, a urea group, a thiourea group, a carbonate group, a carbamate group, an ether bond, or a thioether bond;
P comprises a water soluble polymer chain, a dendrimer, a polysaccharide, a peptide, a protein, a polymer-peptide conjugate, or a polymer-protein conjugate;
n is an integer between 2 and 10,000; and
L comprises diethylenetriaminepentaacetate (DTPA) or its derivatives; 1,4,7,10-tetraazadodecanetetra-acetate (DOTA) or its derivatives; 1,4,7,10-tetraazadodecane-1,4,7-triacetate (DO3A) or its derivatives, or a chelating ligand.

4. The method of claim 1, wherein the macromolecular Gd(III) complex comprises the formula III 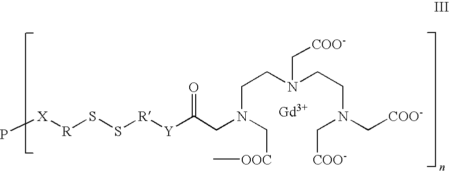
wherein R and R′ comprises, independently, a C₁ to C₁₈ alkyl group, a substituted alkyl group, an unsubstituted aryl group or an aryl group substituted with one or more functional groups comprising an alkyl group, an aryl group, a polyethylene glycol, a saccharide, an amino acid, a peptide, a protein, a peptide conjugate, or a protein conjugate;
X comprises an amide group, an ester group, a urea group, a thiourea group, a carbonate group, a carbamate group, an ether bond, or a thioether bond;
Y comprises O or NH;
n is an integer between 2 and 10,000; and
P comprises a water soluble polymer chain, a dendrimer, a polysaccharide, a peptide, a protein, a polymer-peptide conjugate, or a polymer-protein conjugate.

5. The method of claim 1, wherein the macromolecular Gd(III) complex comprises the formula IV 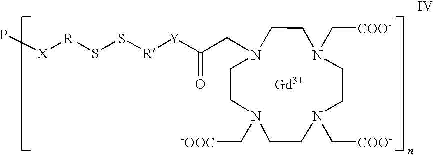
wherein R and R′ comprises, independently, a C₁ to C₁₈ alkyl group, a substituted alkyl group, an unsubstituted aryl group or an aryl group substituted with one or more functional groups comprising an alkyl group, an aryl group, a polyethylene glycol, a saccharide, an amino acid, a peptide, a protein, a peptide conjugate, or a protein conjugate;
X comprises an amide group, an ester group, a urea group, a thiourea group, a carbonate group, a carbamate group, an ether bond, or a thioether bond;
Y comprises O or NH;
n is an integer between 2 and 10,000; and
P comprises a water soluble polymer chain, a dendrimer, a polysaccharide, a peptide, a protein, a polymer-peptide conjugate, or a polymer-protein conjugate.

6. The method of claim 1, wherein the macromolecular Gd(III) complex comprises the formula V 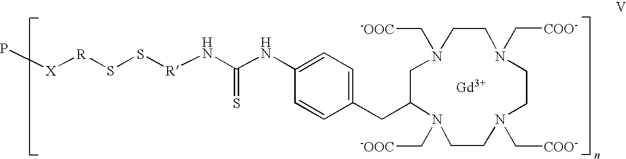
wherein R and R′ comprises, independently, a C₁ to C₁₈ alkyl group, a substituted alkyl group, an unsubstituted aryl group or an aryl group substituted with one or more functional groups comprising an alkyl group, an aryl group, a polyethylene glycol, a saccharide, an amino acid, a peptide, a protein, a peptide conjugate, or a protein conjugate;
X comprises an amide group, an ester group, a urea group, a thiourea group, a carbonate group, a carbamate group, an ether bond, or a thioether bond;
n is an integer between 2 and 10,000; and
P comprises a water soluble polymer chain, a dendrimer, a polysaccharide, a peptide, a protein, a polymer-peptide conjugate, or a polymer-protein conjugate.

7. The method of claim 1, wherein the macromolecular Gd(III) complex comprises the formula VI 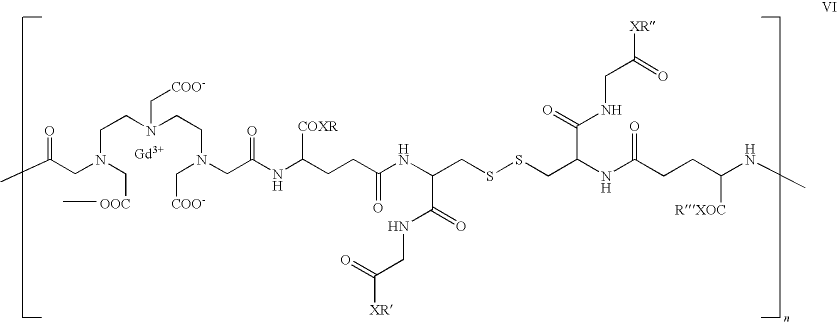
wherein R, R′, R″ and R′″ comprise, independently, a C₁ to C₁₈ alkyl group, a substituted alkyl group, an unsubstituted aryl group or an aryl group substituted with one or more functional groups comprising an alkyl group, an aryl group, a polyethylene glycol, a saccharide, an amino acid, a peptide, a protein, a peptide conjugate, or a protein conjugate;
X comprises O or NH; and
n is an integer between 2 and 10,000.

8. The method of claim 1, wherein the macromolecular Gd(III) complex comprises the formula VII 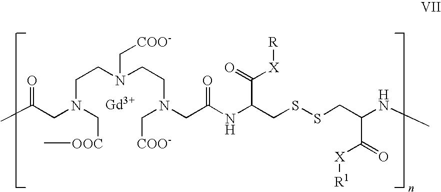
wherein R and R′ comprises, independently, hydrogen, a C₁ to C₁₈ alkyl group, a substituted alkyl group, an unsubstituted aryl group or an aryl group substituted with one or more functional groups comprising an alkyl group, an aryl group, a polyethylene glycol, a saccharide, an amino acid, a peptide, a protein, a peptide conjugate, or a protein conjugate;
X comprises O or NH; and
n is an integer between 2 and 10,000.

9. The method of claim 1, wherein the macromolecular Gd(III) complex comprises the formula VIII 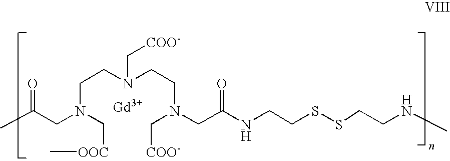
wherein n is an integer between 2 and 10,000.

10. The method of claim 1, wherein the contrast agent is administered to the subject by intravenous injection.

11. The method of claim 1, wherein the contrast agent is administered to the subject in an amount of 0.001 to 0.5 mmol-Gd/kg.

12. The method of claim 1, wherein step (b) comprises measuring the signal intensity of the contrast agent at a region of interest in the tumor.

13. The method of claim 12, wherein the region of interest comprises the periphery of the tumor, one or more specific regions in the tumor, or whole tumor.

14. The method of claim 1, wherein the tumor property comprises tumor size, tumor microvascular characteristics, fractional tumor blood volume, flow leakage rate, permeability surface area, vascular permeability, or transfer constant.

15. The method of claim 1, wherein the treatment comprises administering to the subject an anti-cancer agent.

16. The method of claim 1, wherein the treatment comprises administering to the subject an anti-cancer agent and subsequently detecting the signal intensity of the contrast agent at the region of interest in the tumor, wherein a decrease in signal intensity compared to the baseline signal intensity at the region of interest in the tumor indicates decreased blood flow within the tumor.

17. The method of claim 1, wherein the treatment comprises administering to the subject an anti-cancer agent and subsequently quantifying the signal intensity of the contrast agent at the region of interest in the tumor, wherein a decrease in signal intensity compared to the baseline signal intensity at the region of interest in the tumor indicates decreased blood flow within the tumor.

18. The method of claim 15, wherein the anti-cancer agent comprises an anti-angiogenic agent.

19. The method of claim 1, wherein the treatment step comprises photodynamic therapy of cancer.

20. The method of claim 19, wherein the treatment step comprises thermal ablation, or radiofrequency ablation, cryoablation, high intensity focused ultrasound ablation (HIFU), or laser ablation.

21. The method of claim 1, wherein the magnetic resonance imaging comprises dynamic contrast-enhanced MRI.

22. The method of claim 1, wherein steps (d) and (e) are performed multiple times over time to evaluate the treatment of the tumor over time.
